(19) United States
(12) Patent Application Publication
(10) Pub. No.: US 2024/0389984 A1
(43) Pub. Date: Nov. 28, 2024

(54) TWO COMPARTMENT FACE MASK

(71) Applicant: 3M Innovative Properties Company, Saint Paul, MN (US)

(72) Inventor: Michael Wohl, Bethesda, MD (US)

(21) Appl. No.: 18/691,642

(22) PCT Filed: Sep. 16, 2022
(86) PCT No.: PCT/US22/43827

§ 371 Date: Mar. 13, 2024

Related U.S. Application Data
(60) Provisional application No. 63/245,021, filed on Sep. 16, 2021.

(51) Int. Cl. A61B 10/00; A41D 13/11; A62B 23/02

(52) U.S. Cl. CPC A61B 10/0045; A41D 13/1107; A62B 23/025; A61B 2010/0087

(57) ABSTRACT

Disclosed are filtering face masks. The face mask includes a mask body having an upper portion of a first material and configured to cover a user's nostrils: a lower portion of a second material configured to cover the user's mouth; and a divider configured to separate the upper portion and the lower portion. The first material, second material, or both include a filtration material. The face mask may include a sample collection material. The sample collection material may be provided as an insert.

### RELATED APPLICATIONS

This application claims priority to U.S. Provisional Patent Application No. 63/245,021, filed on Sep. 16, 2021, which is incorporated herein in its entirety.

### BACKGROUND

Respirators are commonly worn over a person's breathing passages for at least one of two common purposes: (1) to prevent impurities or contaminants from entering the user's respiratory system; and (2) to protect other persons or things from being exposed to pathogens and other contaminants exhaled by the user. In the first situation, the respirator is worn in an environment where the air contains particles (e.g., pathogens) that are harmful to the user. In the second situation, the respirator is worn in an environment where there is risk of contamination to other persons or things, for example, to protects others from pathogens present in the user's body.

A variety of respirators have been designed to meet either (or both) of these purposes. Some respirators have been categorized as being “filtering face-pieces” because the mask body itself functions as the filtering mechanism. Unlike respirators that use rubber or elastomeric mask bodies in conjunction with attachable filter cartridges (such as those described in U.S. Patent RE39,493 to Yuschak et al.) or insert-molded filter elements (such as those described in U.S. Pat. No. 4,790,306 to Braun), filtering face-piece respirators are designed to have the filter media cover much of the whole mask body so that there is no need for installing or replacing a filter cartridge. These filtering face-piece respirators commonly come in one of two configurations: molded respirators and flat-fold respirators.

Molded filtering face piece respirators have regularly comprised non-woven webs of thermally-bonding fibers or open-work plastic meshes to furnish the mask body with its cup-shaped configuration. Molded respirators tend to maintain the same shape during both use and storage. These respirators therefore cannot be folded flat for storage and shipping. Examples of patents that disclose molded, filtering, face-piece respirators include U.S. Pat. No. 7,131,442 to Kronzer et al, U.S. Pat. Nos. 6,923,182, 6,041,782 to Angadjivand et al., U.S. Pat. No. 4,807,619 to Dyrud et al., and U.S. Pat. No. 4,536,440 to Berg.

Flat-fold respirators—as their name implies—can be folded flat for shipping and storage. They also can be opened into a cup-shaped configuration for use. Examples of flat-fold respirators are shown in U.S. Pat. Nos. 6,568,392 and 6,484,722 to Bostock et al., and U.S. Pat. No. 6,394,090 to Chen.

### SUMMARY

The present disclosure generally relates to medicine, as well as overall personal, industrial, and environmental hygiene. More particularly, the present disclosure is directed to filtering face masks. The filtering face masks may be used to protect a user from airborne pathogens and other airborne contaminants. The filtering face masks may be used to protect others from pathogens present in the user's body that may become airborne upon exhalation.

In one aspect, the present disclosure is directed to a face mask including an upper portion of a first material configured to cover a user's nostrils, a lower portion of a second material configured to cover the user's mouth; and a divider configured to separate the upper portion and the lower portion. The first material, second material, or both include a filtration material. The face mask may include a sample collection material. The sample collection material may be provided as an insert.

Research suggest that respiratory pathogens are spread predominantly by breath exhaled through the nostrils. However, breathability of a face mask is a major concern to users and a main factor for use-compliance. People looking for self-protection and to protect others will seek out masks that filter the most containments while being easy to breathe through. Air flow, temperature and humidity breathed by an individual affects user experience when wearing a mask.

Pathogens, such as COVID, can attack the body through ACE2 receptors found in the nasal cavity. The nasal cavity needs to be particularly protected from pathogens entering in this manner.

Accordingly, there exists a continuing need to develop face masks that can reduce or prevent the spread of pathogens as well as improve breathability to increase mask use.

### BRIEF DESCRIPTION OF FIGURES

FIG. 1A is a perspective view of a filtering face mask according to an embodiment.

FIG. 1B is a side cross-sectional view of the filtering face mask of FIG. 1A.

FIG. 1C is a top cross-sectional view of the filtering face mask of FIG. 1A.

FIG. 2 is a plan view of a divider for the filtering face mask of FIG. 1A.

FIG. 3 is a plan view of a divider with an insert for the filtering face mask of FIG. 1A.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

All headings provided herein are for the convenience of the reader and should not be used to limit the meaning of any text that follows the heading, unless so specified.

The term “i.e.” is used here as an abbreviation for the Latin phrase id est, and means “that is,” while “e.g.” is used as an abbreviation for the Latin phrase exempli gratia and means “for example.”

The term “about” is used here in conjunction with numeric values to include normal variations in measurements as expected by persons skilled in the art and is understood have the same meaning as “approximately” and to cover a typical margin of error, such as ±5% of the stated value. Moreover, unless otherwise indicated, all numbers expressing quantities, and all terms expressing direction/orientation (e.g., vertical, horizontal, parallel, perpendicular, etc.) in the specification and claims are to be understood as being modified in all instances by the term “about.”

Terms such as “a,” “an,” and “the” are not intended to refer to only a singular entity but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have”, “having”, “include”, “including”, “comprise”, “comprising” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method or the like, means that the components of the composition, product, method or the like are limited to the enumerated components and any other components that do not materially affect the basic and novel characteristic(s) of the composition, product, method or the like.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to here, such as “front,” “back,” “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

The terms “downstream” and “upstream” refer to a relative position based on a direction of exhalation airflow through the device. For example, the upstream-most element of the device is the air inlet element, and the downstream-most element of the device is the exhalation outlet element.

### DETAILED DESCRIPTION

Disclosed herein are filtering face masks (i.e., respiratory protective devices). The filtering face masks of the present disclosure may be effective for filtering pathogens, such as viruses, from exhaled or inhaled air. The filtering face masks of the present disclosure may further be capable of capturing a sample from exhaled air that may be used for testing.

Research indicates that some respiratory pathogens are spread predominantly by breath exhaled through the nostrils. Pathogens, such as the COVID virus, can attack the body through ACE2 receptors found in the nasal cavity. There is thus a need to both protect the nasal cavity from pathogens entering from the outside, but also to protect others from pathogens possibly exiting the nasal cavity of an infected person.

Filter materials traditionally used in face masks, such as electrets, perform best when the air is slower moving and the electrical charges in the electret material perform better by attracting and capturing pathogens. Momentum of exhaled breath when decreased increases electret capture performance.

Breathability of a face mask is a major concern to users and a main factor for use-compliance. People are more likely to wear a face mask that allows for comfortable breathing. People looking for self protection and to protect others will seek out masks that filter the most containments while being easy to breathe through.

According to an embodiment, the filtering face masks disclosed herein provide a close facial fit and efficient filtration of airborne particles. The face mask includes an upper portion (forming an upper compartment), a lower portion (forming a lower compartment), and a divider that separates the upper portion and the lower portion. The upper portion is made of a first material and the lower portion is made of a second material. In some embodiments, the second material is different from the first material. The divider may be made of the first material or the second material or a third material that is different from the first and second materials. In some embodiments, the filtering face mask also includes a sample collection material. In some embodiments, the face mask is constructed as a flat-fold mask. In some embodiments, the face mask is constructed as a molded mask.

According to an embodiment, the upper portion provides a close facial fit of a user's nostrils. The upper portion may be designed to form a seal with the surface of the user's skin to reduce or prevent air from flowing through gaps or openings between the user's skin surface and the mask. Forming a close facial fit against the user's skin may help direct air flow through the mask material.

According to an embodiment, the upper portion is made of a first material and the lower portion is made of a second material. The first and second materials may be the same material or different materials. In some embodiments, the second material is different from the first material. The first material may be less porous and/or less dense than second material. The first material may have a higher pressure drop than the second material. The divider may be made of the first material or the second material or a third material that is different from the first and second materials. In some embodiments, the divider is made of the second material or a third material that also has a lower pressure drop than the first material.

Reference to pressure drop here is intended to mean pressure drop as measured according to NIOSH-42CFR84 at a flow rate of 85 L/min. It should be noted that the pressure drop across any filter material will naturally be higher at higher flow rates and lower at lower flow rates. To the extent that a material may have a different exhalation and inhalation pressure drop, reference is made here to exhalation pressure drop. Filtration materials suitable for use in filtering face masks, such as those described here, typically have pressure drop in the range of 10 Pa to 400 Pa at a flow rate of 85 L/min. For example, an N95 material according to the NIOSH standard has a maximum exhalation pressure drop of equal to or less than 245 Pa and a maximum inhalation pressure drop of equal to or less than 343 Pa at a flow rate of 85 L/min.

According to an embodiment, the first material and second material have pressure drops in the range of 10 Pa to 300 Pa, 10 Pa to 200 Pa, 10 Pa to 100 Pa, 10 Pa to 50 Pa, 10 Pa to 40 Pa, 12 Pa to 25 Pa, 10 Pa to 20 Pa, or 12 Pa to 20 Pa. Within the overall range, the first material used to construct the upper portion may have a higher pressure drop than the second material used to construct the lower portion. For example, in some embodiments, the first material has a pressure drop of 10 Pa to 300 Pa, 10 Pa to 200 Pa, 12 Pa to 100 Pa, 12 Pa to 50 Pa, 12 Pa to 40 Pa, 12 Pa to 25 Pa, or 12 Pa to 20 Pa. The second material may have a pressure drop that is lower than that of the first material. The second material may have a pressure drop of 10 Pa to 300 Pa, 10 Pa to 200 Pa, 10 Pa to 100 Pa, 10 Pa to 50 Pa, 10 Pa to 40 Pa, 10 Pa to 25 Pa, or 10 Pa to 20 Pa. In some embodiments, the first material used to make the upper portion is an N95-like material (including N95 material). The pressure drop of the various materials may be selected to guide airflow in a desired pattern and to improve sample collection at the sample collection material. For example, the pressure drop of the lower portion and a sample collection material insert in the divider may be lower than the upper portion and the rest of the divider such that air more easily flows through the sample collection material and the lower portion. Making the lower portion of a lighter, more breathable material may also increase user comfort by making mouth breathing easier and allowing cooler filtered air to enter upper compartment. Making the upper portion of a heavier, higher pressure drop material slows down the airflow from nose breathing and forces more air to pass through the divider and sample collection material, thus improving capture by the sample collection material. Air inhaled through the divider and the bottom portion is double filtered as it passes through at least two layers of material.

The first material, second material, or both the first and second materials may be layered materials. According to an embodiment, the first and second materials include at least a filtration layer. The filtration layer is typically chosen to achieve a desired filtering effect. The filtration layer generally will remove a high percentage of particles and/or or other contaminants from the gaseous stream that passes through it. For fibrous filter layers, the fibers selected depend upon the kind of substance to be filtered and, typically, are chosen so that they do not become bonded together during the molding operation. As indicated, the filtration layer may come in a variety of shapes and forms and typically has a thickness of about 0.2 millimeters (mm) to 1 centimeter (cm), more typically about 0.3 mm to 0.5 cm, and it could be a generally planar web or it could be corrugated to provide an expanded surface area—see, for example, U.S. Pat. Nos. 5,804,295 and 5,656,368 to Braun et al. The filtration layer also may include multiple filtration layers joined together by an adhesive or any other means. Essentially any suitable material that is known (or later developed) for forming a filtering layer may be used as the filtering material. Webs of melt-blown fibers, such as those taught in Wente, Van A., Superfine Thermoplastic Fibers, 48 Indus. Engn. Chem., 1342 et seq. (1956), especially when in a persistent electrically charged (electret) form are especially useful (see, for example, U.S. Pat. No. 4,215,682 to Kubik et al.). These melt-blown fibers may be microfibers that have an effective fiber diameter less than about 20 micrometers (μm) (referred to as BMF for “blown microfiber”), typically about 1 to 12 μm. Effective fiber diameter may be determined according to Davies, C. N., The Separation Of Airborne Dust Particles, Institution Of Mechanical Engineers, London, Proceedings 1B, 1952. Particularly preferred are BMF webs that contain fibers formed from polypropylene, poly (4-methyl-1-pentene), and combinations thereof. Electrically charged fibrillated-film fibers as taught in van Turnhout, U.S. Patent Re. 31,285, also may be suitable, as well as rosin-wool fibrous webs and webs of glass fibers or solution-blown, or electrostatically sprayed fibers, especially in microfilm form. Electric charge can be imparted to the fibers by contacting the fibers with water as disclosed in U.S. Pat. No. 6,824,718 to Eitzman et al., U.S. Pat. No. 6,783,574 to Angadjivand et al., U.S. Pat. No. 6,743,464 to Insley et al., U.S. Pat. Nos. 6,454,986 and 6,406,657 to Eitzman et al., and U.S. Pat. Nos. 6,375,886 and 5,496,507 to Angadjivand et al. Electric charge also may be imparted to the fibers by corona charging as disclosed in U.S. Pat. No. 4,588,537 to Klasse et al. or by tribocharging as disclosed in U.S. Pat. No. 4,798,850 to Brown. Also, additives can be included in the fibers to enhance the filtration performance of webs produced through the hydro-charging process (see U.S. Pat. No. 5,908,598 to Rousseau et al.). Fluorine atoms, in particular, can be disposed at the surface of the fibers in the filter layer to improve filtration performance in an oily mist environment—see U.S. Pat. Nos. 6,398,847 B1, 6,397,458 B1, and 6,409,806 B1 to Jones et al. Typical basis weights for electret BMF filtration layers are about 10 to 100 grams per square meter. When electrically charged according to techniques described in, for example, the '507 Angadjivand et al. patent, and when including fluorine atoms as mentioned in the Jones et al. patents, the basis weight may be about 20 to 40 g/m² and about 10 to 30 g/m², respectively.

One or both of the first and second materials may further include a pre-filter layer. The pre-filter layer may be layer disposed adjacent to the filtration layer and may provide additional filtration capabilities. In some embodiments, the pre-filter layer is disposed between the user's face and the filtration layer. In some embodiments, the pre-filter layer is disposed on the outside of the filtration layer when observed from the user's point of view. Additionally, sorptive materials such as activated carbon may be disposed between the fibers and/or various layers that comprise the filtering structure. Further, separate particulate filtration layers may be used in conjunction with sorptive layers to provide filtration for both particulates and vapors. The filtering structure may include one or more stiffening layers that assist in providing a cup-shaped configuration.

One or both of the first and second materials may further include a support layer, such as a cover web. For example, An inner cover web can be used to provide a smooth surface for contacting the user's face, and an outer cover web can be used to entrap loose fibers in the mask body or for aesthetic reasons. The cover web typically does not provide any substantial filtering benefits to the filtering structure, although it can act as a pre-filter when disposed on the exterior (or upstream to) the filtration layer. To obtain a suitable degree of comfort, an inner cover web preferably has a comparatively low basis weight and is formed from comparatively fine fibers. More particularly, the cover web may be fashioned to have a basis weight of about 5 to 50 g/m² (typically 10 to 30 g/m²), and the fibers may be less than 3.5 denier (typically less than 2 denier, and more typically less than 1 denier but greater than 0.1). Fibers used in the cover web often have an average fiber diameter of about 5 to 24 micrometers, typically of about 7 to 18 micrometers, and more typically of about 8 to 12 micrometers. The cover web material may have a degree of elasticity (typically, but not necessarily, 100 to 200% at break) and may be plastically deformable.

Suitable materials for the cover web may be blown microfiber (BMF) materials, particularly polyolefin BMF materials, for example polypropylene BMF materials (including polypropylene blends and also blends of polypropylene and polyethylene). A suitable process for producing BMF materials for a cover web is described in U.S. Pat. No. 4,013,816 to Sabee et al. The web may be formed by collecting the fibers on a smooth surface, typically a smooth-surfaced drum or a rotating collector—see U.S. Pat. No. 6,492,286 to Berrigan et al. Spun-bond fibers also may be used.

A typical cover web may be made from polypropylene or a polypropylene/polyolefin blend that contains 50 weight percent or more polypropylene. These materials have been found to offer high degrees of softness and comfort to the user and also, when the filter material is a polypropylene BMF material, to remain secured to the filter material without requiring an adhesive between the layers. Polyolefin materials that are suitable for use in a cover web may include, for example, a single polypropylene, blends of two polypropylenes, and blends of polypropylene and polyethylene, blends of polypropylene and poly (4-methyl-1-pentene), and/or blends of polypropylene and polybutylene. One example of a fiber for the cover web is a polypropylene BMF made from the polypropylene resin “Escorene 3505G” from Exxon Corporation, providing a basis weight of about 25 g/m² and having a fiber denier in the range 0.2 to 3.1 (with an average, measured over 100 fibers of about 0.8). Another suitable fiber is a polypropylene/polyethylene BMF (produced from a mixture comprising 85 percent of the resin “Escorene 3505G” and 15 percent of the ethylene/alpha-olefin copolymer “Exact 4023” also from Exxon Corporation) providing a basis weight of about 25 g/m² and having an average fiber denier of about 0.8. Suitable spunbond materials are available, under the trade designations “Corosoft Plus 20”, “Corosoft Classic 20” and “Corovin PP-S-14”, from Corovin GmbH of Peine, Germany, and a carded polypropylene/viscose material available, under the trade designation “370/15”, from J.W. Suominen OY of Nakkila, Finland.

Cover webs that are used in the face mask may have very few fibers protruding from the web surface after processing and therefore have a smooth outer surface. Examples of cover webs that may be used in the present invention are disclosed, for example, in U.S. Pat. No. 6,041,782 to Angadjivand, U.S. Pat. No. 6,123,077 to Bostock et al., and WO 96/28216A to Bostock et al.

The lower portion of the mask covers a user's mouth. The lower portion of the mask is made of a second material that is more porous than the first material used to make the upper portion of the mask. This arrangement makes the lower portion more breathable to the user. It may also provide improved temperature control, fresh air conveyance, and improved audibility while speaking.

The face mask also includes a divider that forms a barrier between the nostrils and the mouth, and thus, defines the upper portion (compartment) and the lower portion (compartment) of the mask. The divider may be made of the same material as the upper portion or lower portion, or a different material than either the upper or the lower portion. The divider may be made of a permeable or non-permeable material. In some embodiments, the divider is made of heavier pressure drop material than the lower portion. The divider may include an insert including sample collection material. The sample collection material may have a lesser pressure drop so that air flows through the sample collection material to facilitate pathogen collection. The insert may be removed for pathogen testing.

The divider may be positioned on the mask body such that when the mask is worn, the divider is positioned below the nose. The divider may be positioned above the mouth. In some embodiments, the divider is positioned below the mouth. The mask body may have a height, and the divider may be placed at a location measured as a percentage of the height measured from the top of the mask body. For example, the divider may be placed at least 20% of the height from the top of the mask, at least 25% of the height, at least 30% of the height, or at least 40% of the height. The divider may be placed or from 20% to 50% of the height, or from 25% to 40% of the height. The divider may be placed at about 30% of the height, about 35% of the height, or about 40% of the height. In some embodiments, where the divider is intended to be positioned below the mouth, the divider may be positioned at or below about 50% of the height.

The divider may extend from one side of the mask to the opposing side of the mask. The divider may extend across only part of the width of the mask. For example, the divider may extend across the middle 50% or greater, 75% or greater, or 90% or greater, of the width of the mask.

The divider may have a depth (front to back) that accommodates a majority of facial shapes and sizes. The depth of the divider extends from the mask body (positioned in front of the user's face) to the user's face.

In some embodiments, the face mask is constructed as a flat-fold mask. The flat-fold mask may be folded with the divider in place. The flat-fold mask may be folded with the insert in place. Alternatively, the flat-fold mask may be folded without the insert and the insert may be inserted prior to use. The flat-fold mask may be provided as a kit including an insert and instructions for unfolding the mask and inserting the insert.

The face mask may further include exhalation sample collection material. For example, the upper portion, the lower portion, the divider, or a combination thereof may include exhalation sample collection material. The sample collection material may be provided as a removable insert. In some embodiments, the upper portion or the divider of the face mask includes a material designed to capture and collect analytes in a bio-aerosol sample expelled through the user's nostrils. In one embodiment, the entire upper portion of the mask can capture and collect analytes in a bio-aerosol sample. In one embodiment, a region of the upper portion of the mask can capture and collect analytes in a bio-aerosol sample. In one embodiment a separate material that functions to capture and collect analytes in a bio-aerosol sample is coupled or attached to the mask on the interior surface (the surface of the mask that faces the user's face). The sample collection material may be removably coupled with or attached to the mask. In one embodiment, the divider can capture and collect analytes in a bio-aerosol sample. In one embodiment, a portion of the divider can capture and collect analytes in a bio-aerosol sample. In one embodiment, the divider can include a separate material that is coupled or attached to the divider that can capture and collect analytes in a bio-aerosol sample. The second material can be separated from upper portion of the mask and/or the divider and analyzed.

The sample collection material may have any suitable size. The size of the sample collection material may be a size that facilitates inhalation through the sample collection material. For example, if the pressure drop through the sample collection material is different than the other materials used to make the mask, the size of the sample collection material may be such that it facilitates a suitable over-all pressure drop through the mask. The size of the sample collection material may be a size that facilitates collection of a samples from airflow passing through the sample collection material. The size of the sample collection material may be a size that facilitates attachment of the sample collection material to the mask. The sample collection material may be an insert or may form one or more of the parts of the mask, such as the divider, the upper portion, the lower portion, or a combination thereof. When the sample collection material is provided as an insert, the size of the sample collection material may be smaller than when the sample collection material is provided as, for example, the divider. The surface area of the sample collection material is understood as the surface area of a single major surface of the material. The sample collection material may have a major surface having a surface area of 50 mm² or greater, 100 mm² or greater, 200 mm² or greater, or 300 mm² or greater. The surface are of the major surface may be 500 mm² or less, 400 mm² or less, 300 mm² or less, or 250 mm² or less. The surface are of the major surface may be from 50 mm² to 500 mm², 75 mm² to 400 mm², or 100 mm² to 300 mm².

The sample collection material may be an insert that covers a window or opening in the mask. For example, the mask may include a window or opening in the divider or the lower portion or both. In one embodiment, the mask includes one or more windows or openings in the divider. The one or more windows or openings may be positioned directly below the nose or nostrils. Alternatively, the one or more windows or openings may be positioned at the sides of the divider. The one or more windows or openings may be covered by the sample collection material. The sample collection material may be removable and may be used for testing for virus, other pathogens, or other analytes.

In another embodiment, the entire upper portion of the mask and/or the entire divider can be made of a material that attracts analytes contained in the bio-aerosol sample expelled from the user's nostril(s) or filtered from the exterior air via inhalation. The entire upper portion of the mask, a portion of the upper portion of the mask, the entire divider, or a portion of the divider can be analyzed for an analyte of interest.

A particularly suitable material to capture and collect a sample is electret. In another embodiment, a sample capture and collection electret material can be coupled or attached to the upper portion of the mask and/or the divider. The sample capture and collection electret material can be removed from the mask or divider and analyzed.

According to an embodiment, the sample collection material is a nonwoven material carrying an electrostatic charge. The electrostatic charge may enable capturing pathogens, viruses, or other analytes from an exhalation airflow. In some cases, the sample collection material may be a hydrophobic nonwoven material. In other cases, the sample collection material may be a hydrophilic nonwoven material. The sample collection material may be a hydrophobic nonwoven material carrying an electrostatic charge configured to capture pathogens, viruses, or other analytes from an exhalation airflow. The sample collection material may be a hydrophilic nonwoven material carrying an electrostatic charge configured to capture pathogens, viruses, or other analytes from an exhalation airflow. The term “hydrophobic” refers to a material having a water contact angle of 90 degrees or greater, or from about 90 degrees to about 170 degrees, or from about 100 degrees to about 150 degrees. The term “hydrophilic” refers to a material having a water contact angle of less than 90 degrees. Water contact angle is measured using ASTM D5727-1997 Standard test method for surface wettability and absorbency of sheeted material using an automated contact angle tester.

The sample collection material may be formed of any suitable material that is capable of capturing viruses, pathogens, or other analytes from exhalation airflow and releasing the captured viruses, pathogens, or other analytes upon being contacted with an eluent, such as a saline solution. The sample collection material may be formed of polymeric material. The sample collection material may be formed of a polyolefin. Examples of suitable polyolefins include polypropylene, polylactic acid, and the like, and a combination thereof. In one embodiment the sample collection material is formed of polypropylene. In one embodiment the sample collection material is formed of polylactic acid. One illustrative sample collection material is commercially available from 3M Company (St. Paul MN, U.S.A.) under the trade designation FILTRETE Smart MPR 1900 Premium Allergen, Bacteria & Virus Air Filter Merv 13.

The sample collection material may have a thickness (orthogonal to the major plane) of 200 μm or greater or 250 μm or greater. The sample collection material may have a thickness of 750 μm or less or 1000 μm or less. The sample collection material may have a thickness of in a range from 200 μm to 1000 μm, or from 250 μm to 750 μm.

The user may exhale into and through the mask to load the sample collection material with a sample of the exhalation airflow to form a loaded sample collection material. For example, the user may exhale through the nostrils and through the upper portion of the mask such that the exhalation airflow passes through the sample collection material. The sample collection material may also be used to test for exposure to viruses or other pathogen's in the environment. The user may inhale air such that the inhalation airflow passes through the sample collection material. The sample collection material is constructed to capture viruses, other pathogens, or other analytes, from the exhalation airflow, inhalation airflow, or both. The user may later remove the portion of the mask containing the loaded sample collection material. The user may use a suitable assay to test the loaded sample collection material for the presence of a pathogen, or may supply the loaded sample collection material to a testing facility or health care facility for testing. The sample may be eluted from the loaded sample collection material using a suitable liquid, such as a buffer, and be further tested using any suitable assay. In some embodiments, the mask may be used in a setting where the user may be exposed to a pathogen (e.g., respiratory virus) or multiple such pathogens and after a period of exposure, the sample collection material may be removed and submitted for testing. For example, the mask may be worn in a healthcare setting, education setting, hospitality setting, restaurants, travel setting, etc. In one exemplary embodiment, a healthcare worker may wear the mask for the duration of a work shift and have the sample collection insert tested at the end of the work shift.

The lower portion may be permanently or removably attached to the upper portion. In one embodiment, the lower portion is adjustable and/or removable. In this embodiment, the upper portion and divider can remain in place, covering the nose and nostrils, while the user lowers, raises, or removes the lower portion of the mask to allow the user to eat, drink, or speak. The mask can include a hinge mechanism to connect the lower portion to the mask that allows for lifting the lower portion up, lowering the lower portion down, and/or removing the lower portion. The mask can include a hook-and-loop fastener, adhesive, or other fastener to connect the lower portion to the mask to allow for oral access.

The face mask may further include additional components to facilitate the use and facial fit of the face mask. For example, the face mask may include straps, a nose clip, a seal, an exhalation valve, or a combination thereof.

The strap(s) that are used in the face mask may be made from a variety of materials, such as thermoset rubbers, thermoplastic elastomers, braided or knitted yarn/rubber combinations, inelastic braided components, and the like. The strap(s) may be made from an elastic material such as an elastic braided material. The strap preferably can be expanded to greater than twice its total length and be returned to its relaxed state. The strap also could possibly be increased to three or four times its relaxed state length and can be returned to its original condition without any damage thereto when the tensile forces are removed. The elastic limit thus is preferably not less than two, three, or four times the length of the strap when in its relaxed state. Typically, the strap(s) are about 20 cm to 30 cm long, 3 mm to 10 mm wide, and about 0.9 mm to 1.5 mm thick. The strap(s) may extend from a first side of the mask to a second side as a continuous strap or the strap may have a plurality of parts, for example loops that may be worn about the ears, or plurality of parts which can be joined together by further fasteners or buckles. For example, the strap may have first and second parts that are joined together by a fastener that can be quickly uncoupled by the user when removing the mask body from the face. An example of a strap that may be used in connection with the present invention is shown in U.S. Pat. No. 6,332,465 to Xue et al. Examples of fastening or clasping mechanism that may be used to joint one or more parts of the strap together is shown, for example, in the following U.S. Pat. No. 6,062,221 to Brostrom et al., U.S. Pat. No. 5,237,986 to Seppala, and EP1,495,785A1 to Chien.

A nose clip may be essentially any additional part that assists in improving the fit over the user's nose. Because the user's face exhibits in the nose region, a nose clip may be used to better assist in achieving the appropriate fit in this location. The nose clip may comprise, for example, a pliable dead soft band of metal such as aluminum, which can be shaped to hold the mask in a desired fitting relationship over the nose of the user and where the nose meets the cheek. An example of a suitable nose clip is shown in U.S. Pat. No. 5,558,089 and Des. 412,573 to Castiglione. Other nose clips are described in U.S. Pat. No. 8,066,006 and 8,171,933.

A sealing material may be provided around all or part of the mask. The sealing material may be any deformable material or structure. The sealing material may be or may include foam, rubber, elastic, ruching, a combination thereof, or any other suitable material or structure that is able to conform to the contour or a user's face. For example, a seal constructed to seal the mask against the user's face may be provided around the perimeter of the mask where the mask comes into contact with the user's face. A seal may be provided along the free edge of the divider. A seal may be provided around the upper portion of the mask. A seal may be provided around the lower portion of the mask.

An exhalation valve may be attached to the mask body to facilitate purging exhaled air from the interior gas space. The use of an exhalation valve may improve user comfort by rapidly removing the warm moist exhaled air from the mask interior. In some embodiments, an exhalation valve is included in the lower portion of the mask. See, for example, U.S. Pat. Nos. 7,188,622, 7,028,689, and 7,013,895 to Martin et al.; U.S. Pat. Nos. 7,428,903, 7,311,104, 7,117,868, 6,854,463, 6,843,248, and 5,325,892 to Japuntich et al.; U.S. Pat. No. 6,883,518 to Mittelstadt et al.; and RE37,974 to Bowers. Essentially any exhalation valve that provides a suitable pressure drop and that can be properly secured to the mask body may be used in connection with the present invention to rapidly deliver exhaled air from the interior gas space to the exterior gas space.

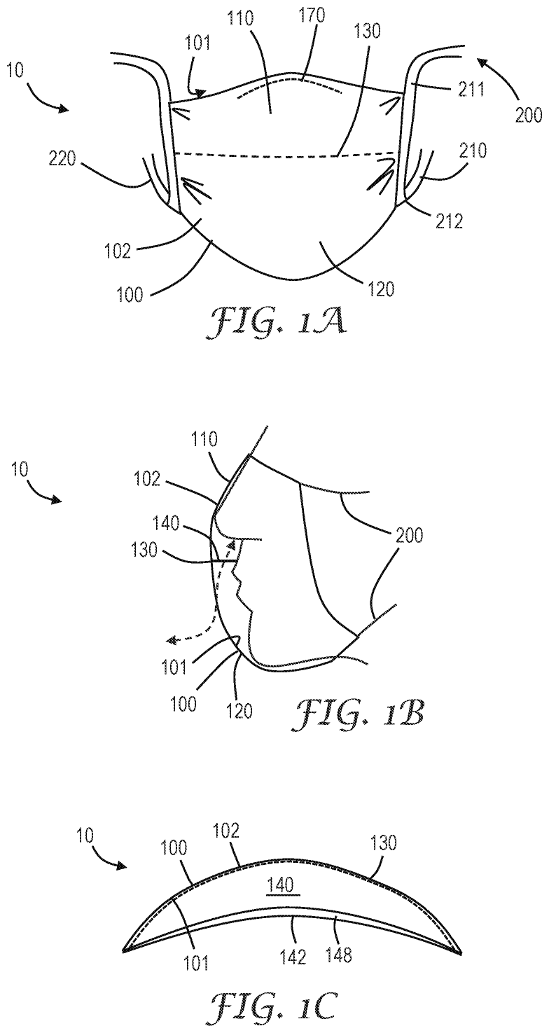

Exemplary embodiments and materials are illustrated in FIGS. 1A-3. As shown in FIG. 1A, the face mask 10 includes a mask body 100 and straps 200. The mask body 100 is made up of an upper portion 110 connected to a lower portion 120. The upper portion 110 and the lower portion 120 may be separated by a line of demarcation 130 extending transversely across the central portion of the face mask body 100 from side to side. In the exemplary embodiment shown, the straps 200 include a first strap 210 extending from a first end 211, attached at an upper corner to the mask body 100, to a second end 212, attached at a lower corner to the mask body 100. A second strap 220 is attached to the other side of the mask body 100 in a similar manner.

The mask body 100 has an inside surface 101 and an outside surface 102. The inside surface 101 faces the user when the mask 10 is worn. The outside surface 102 faces away from the user. FIG. 1B shows a side cross-sectional view of the mask 10. A divider 140 extends from the inside surface 101 along the line of demarcation 130. The area above the divider 140 forms the upper portion 110. According to an embodiment, the user's entire nose, including nostrils, fits within the upper portion 110 when the mask 10 is worn. The upper portion 110 may form a seal against the user's face around the bridge of the nose. The area below the divider 140 forms the lower portion 120. According to an embodiment, the user's mouth and optionally chin fits within the lower portion 120 when the mask 10 is worn. The lower portion 120 may form a seal against the user's face around the mouth.

The mask 10 may include a nose clip 170. The nose clip 170 may be positioned near the top of the upper portion 110 to help improve the facial fit of the mask 10.

The upper portion 110 may be made of a first material and the lower portion 120 may be made of a second material. The second material may be different from the first material. For example, the second material may have a lower pressure drop across the material than the first material, making it easier for a user to breathe through the lower portion 120. Suitable materials for the upper and lower portions 110, 120 are discussed above. The divider 140 may be made of the same material as the upper portion 110, or from a different material. The inside edge 142 of the divider 140 may further include a seal 148 to help improve the fit of the divider 140 against the face of the user.

The divider 140 is shown in FIG. 2. the divider 140 has an outer perimeter 141 and an inside edge 142. The divider 140 has a first major side 144 (e.g., an upper side) and an opposing second major side (e.g., a lower side). The location of the nose 400 and the nostrils 410 on the first major side 144 of the divider 140 is shown in phantom. The divider 140 attaches to the body 100 of the mask along its outer perimeter 141. The outer perimeter 141 may follow the contour of the inside surface 101 of the mask body 100. The divider 140 may be permanently or removably attached. The inside edge 142 of the divider 140 seals against the user's face when the mask 10 is worn.

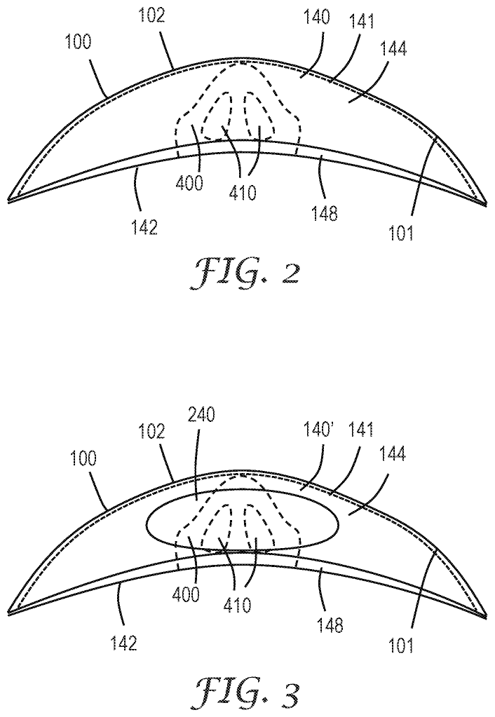

An alternative embodiment of the divider 140′ is shown in FIG. 3, where the divider 140′ includes an insert 240. The insert 240 may be attached onto a major surface of the divider 140′ or may be inserted into a pocket or hole formed in the divider. The insert 240 may be removably attached to the divider 140′. The insert 240 may include sample collection material constructed to collect a sample from exhalation airflow. The insert 240 may be positioned on the divider 140′ so that when the mask is worn, the insert 240 is positioned directly below the nostrils of the user. Although the insert 240 is shown as an oval shape, any suitable shape may be used. The insert 240 may also be the same shape and size as the divider 140′, or may be the same shape but a slightly smaller size than the divider 140′. The insert 240 may be made of a material with a lower pressure drop than the divider 140′ to help guide airflow through the insert 240.

In another alternative embodiment, the entire divider 140 is made of the sample collection material. The entire divider 140 may be removably attached to the mask body 10 such that after use, the loaded sample collection material may be removed for testing.

### Embodiments

The following is a list of exemplary embodiments according to the present disclosure.

Embodiment 1 is a filtering face mask comprising: a mask body having an upper portion of a first material and configured to cover a user's nostrils and a lower portion of a second material coupled with the upper portion and configured to cover the user's mouth, the first material, second material, or both comprising a filtration material; and a divider dividing the mask body into the upper portion and the lower portion.

Embodiment 2 is the filtering face mask of embodiment 1, wherein the first material exhibits a pressure drop that is higher than a pressure drop of the second material, and optionally wherein the divider has a pressure drop that is lower than the pressure drop of the first material, optionally wherein the divider is made of the second material.

Embodiment 3 is the filtering face mask of embodiment 2, wherein the first material has a pressure drop of 10 Pa to 300 Pa, 10 Pa to 200 Pa, 12 Pa to 100 Pa, 12 Pa to 50 Pa, 12 Pa to 40 Pa, 12 Pa to 25 Pa, or 12 Pa to 20 Pa, measured at a flow rate of 85 L/min.

Embodiment 4 is the filtering face mask of embodiment 2 or 3, wherein the second material has a pressure drop of 10 Pa to 300 Pa, 10 Pa to 200 Pa, 10 Pa to 100 Pa, 10 Pa to 50 Pa, 10 Pa to 40 Pa, 10 Pa to 25 Pa, or 10 Pa to 20 Pa, measured at a flow rate of 85 L/min.

Embodiment 5 is the filtering face mask of any one of embodiments 1 to 4 further comprising sample collection material.

Embodiment 6 is the filtering face mask of embodiment 5, wherein the sample collection material comprises a nonwoven filtration layer having an electrostatic charge.

Embodiment 7 is the filtering face mask of embodiment 5 or 6, wherein the nonwoven filtration layer is hydrophobic.

Embodiment 8 is the filtering face mask of any one of embodiments 5 to 7, wherein the sample collection material forms a removable insert.

Embodiment 9 is the filtering face mask of embodiment 8, wherein the removable insert is disposed in or on the divider.

Embodiment 10 is the filtering face mask of any one of embodiments 5 to 9, wherein the sample collection material has a major surface having a surface area of 50 mm² to 500 mm², 75 mm² to 400 mm², or 100 mm² to 300 mm².

Embodiment 11 is the filtering face mask of any one of embodiments 5 to 10, wherein the sample collection material forms at least a part of the upper portion, the lower portion, the divider, or a combination thereof.

Embodiment 12 is the filtering face mask of any one of embodiments 1 to 11, wherein the filtering face mask is a flat-fold mask.

Embodiment 13 is a filtering face mask comprising: a mask body comprising an upper portion and a lower portion, the first portion, second portion, or both comprising a filtration material; and an insert comprising sample collection material.

Embodiment 14 is the filtering face mask of embodiment 13, wherein the upper portion forms an upper compartment configured to fit over a user's nose.

Embodiment 15 is the filtering face mask of embodiment 13 or 14, wherein the insert is disposed within the upper compartment.

Embodiment 16 is the filtering face mask of embodiment 13 or 14, wherein the insert forms a divider dividing the mask body into the upper portion and the lower portion.

Embodiment 17 is the filtering face mask of any one of embodiments 13 to 16, wherein the insert is removably attached to the mask body.

Embodiment 18 is the filtering face mask of any one of embodiments 13 to 17, wherein the sample collection material has a major surface having a surface area of 50 mm² to 500 mm², 75 mm² to 400 mm², or 100 mm² to 300 mm².

Embodiment 19 is the filtering face mask of any one of embodiments 13 to 18, wherein the sample collection material comprises a nonwoven filtration layer having an electrostatic charge.

Embodiment 20 is the filtering face mask of any one of embodiments 13 to 19, wherein the nonwoven filtration layer is hydrophobic.

Embodiment 21 is the filtering face mask of any one of embodiments 13 to 20, wherein first portion is made of a first material and the second portion is made of a second material, and wherein the first material exhibits a pressure drop that is higher than a pressure drop of the second material.

Embodiment 22 is the filtering face mask of any one of embodiments 13 to 21, wherein the first material has a pressure drop of 10 Pa to 300 Pa, 10 Pa to 200 Pa, 12 Pa to 100 Pa, 12 Pa to 50 Pa, 12 Pa to 40 Pa, 12 Pa to 25 Pa, or 12 Pa to 20 Pa, measured at a flow rate of 85 L/min.

Embodiment 23 is the filtering face mask of any one of embodiments 13 to 22, wherein the second material has a pressure drop of 10 Pa to 300 Pa, 10 Pa to 200 Pa, 10 Pa to 100 Pa, 10 Pa to 50 Pa, 10 Pa to 40 Pa, 10 Pa to 25 Pa, or 10 Pa to 20 Pa, measured at a flow rate of 85 L/min.

Embodiment 24 is the filtering face mask of any one of embodiments 13 to 23 further comprising a divider dividing the upper portion and the lower portion.

Embodiment 25 is the filtering face mask of embodiment 24, wherein the divider has a pressure drop that is lower than the pressure drop of the first material, optionally wherein the divider is made of the second material.

Embodiment 26 is the filtering face mask of any one of embodiments 13 to 25, wherein the filtering face mask is a flat-fold mask.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

While the present disclosure is susceptible to various modifications and alternative forms, exemplary embodiments thereof are shown by way of example in the drawings and are herein described in detail. It should be understood, however, that the description of exemplary embodiments is not intended to limit the disclosure to the particular forms disclosed, but on the contrary, the intention is to cover all modifications, equivalents and alternatives falling within the scope of the disclosure as defined by the embodiments above and the claims below. Reference should therefore be made to the embodiments above and claims below for interpreting the scope of the present disclosure.

1. A filtering face mask comprising:
a mask body having an upper portion of a first material and configured to cover a user's nostrils and a lower portion of a second material coupled with the upper portion and configured to cover a user's mouth, the first material, second material, or both comprising a filtration material; and
a divider dividing the mask body into the upper portion and the lower portion.

2. The filtering face mask of claim 1, wherein the first material exhibits a pressure drop that is higher than a pressure drop of the second material.

3. The filtering face mask of claim 1 further comprising sample collection material.

4. The filtering face mask of claim 3, wherein the sample collection material comprises a nonwoven filtration layer having an electrostatic charge.

5. The filtering face mask of claim 4, wherein the nonwoven filtration layer is hydrophobic.

6. The filtering face mask of claim 3, wherein the sample collection material forms a removable insert.

7. The filtering face mask of claim 6, wherein the removable insert is disposed in or on the divider.

8. The filtering face mask of claim 3, wherein the sample collection material has a major surface having a surface area of 50 mm² to 500 mm², 75 mm² to 400 mm², or 100 mm² to 300 mm².

9. The filtering face mask of claim 3, wherein the sample collection material forms at least a part of the upper portion, the lower portion, the divider, or a combination thereof.

10. The filtering face mask of claim 1, wherein the filtering face mask is a flat-fold mask.

11. A filtering face mask comprising:
a mask body comprising an upper portion and a lower portion, the upper portion, lower portion, or both comprising a filtration material; and
an insert comprising sample collection material.

12. The filtering face mask of claim 11, wherein the upper portion forms an upper compartment configured to fit over a user's nose.

13. The filtering face mask of claim 12, wherein the insert is disposed within the upper compartment.

14. The filtering face mask of claim 11, wherein the insert forms a divider dividing the mask body into the upper portion and the lower portion.

15. The filtering face mask of claim 11, wherein the insert is removably attached to the mask body.

16. The filtering face mask of claim 11, wherein the sample collection material has a major surface having a surface area of 50 mm² to 500 mm², 75 mm² to 400 mm², or 100 mm² to 300 mm².

17. The filtering face mask of claim 11, wherein the upper portion is made of a first material and the lower portion is made of a second material, and wherein the first material exhibits a pressure drop that is higher than a pressure drop of the second material.

18. The filtering face mask of claim 11, wherein the sample collection material comprises a nonwoven filtration layer having an electrostatic charge.

19. The filtering face mask of claim 18, wherein the nonwoven filtration layer is hydrophobic.

20. The filtering face mask of claim 11, wherein the filtering face mask is a flat-fold mask.
